(19) United States
(12) Patent Application Publication
(10) Pub. No.: US 2020/0069728 A1
(43) Pub. Date: Mar. 5, 2020

(54) HYPOCHLOROUS ACID BASED EYE DROP AND OPHTHALMIC COMPOSITION

(71) Applicant: Howard J. Loff, Atlantic Beach, FL (US)

(72) Inventor: Howard J. Loff, Atlantic Beach, FL (US)

(21) Appl. No.: 16/674,611

(22) Filed: Nov. 5, 2019

Related U.S. Application Data
(63) Continuation of application No. 62/772,523, filed on Nov. 28, 2018.

(51) Int. Cl. A61K 33/20; A61K 9/00; A01N 25/04; A01N 25/22; A01N 59/00; A61K 9/06

(52) U.S. Cl. CPC A61K 33/20; A61K 9/0048; A61K 9/06; A01N 25/22; A01N 59/00; A01N 25/04

(57) ABSTRACT

The embodiments herein disclose a hypochlorous acid based ophthalmic composition, comprising stabilized hypochlorous acid. The stabilized hypochlorous acid is added in the ophthalmic composition as an anti-microbial agent and/or a preservative. The stabilized hypochlorous acid is added in the ophthalmic composition to be utilized for contact lens solution. The stabilized hypochlorous acid is added in the ophthalmic composition in a non-toxic predetermined amount. The non toxic predetermined amount of stabilized hypochlorous acid less than 0.05% v/v. The stabilized hypochlorous acid is added in the ophthalmic composition as a preservative in a predetermined amount ranging 0.0005% v/v-0.05% v/v. The stabilized hypochlorous acid is added in the ophthalmic composition as an anti-microbial agent after determining free available chlorine. The free available chlorine in the stabilized hypochlorous acid is in a range of 50 ppm-500 ppm. The predetermined ophthalmic compositions are selected from a group consisting of eyedrop solution, eyedrop suspension, eyedrop gel and ointment. anti-microbial eye drops, eye cleansing drops, ophthalmic lubricants and contact lens solutions.

### CROSS-REFERENCE TO RELATED APPLICATIONS

This application claims the priority of U.S. Provisional Patent Application No. 62/772523, filed on Nov. 28, 2018, with the tile, “Novel Use of Hypochlorous Acid As An Eye Drop And Ophthalmic Preservative”, and the content of which is incorporated in its entirety by reference herein.

### BACKGROUND

### Technical Field

The embodiments herein are generally related to a field of pharmaceutical compositions. The embodiments herein are particularly related to eyedrop and ophthalmic compositions. The embodiments herein are more particularly related to stabilized Hypochlorous Acid with enhanced shelf life. The embodiments herein are especially related to an ophthalmic composition incorporating stabilized hypochlorous acid with enhanced shelf life as an antimicrobial agent. The embodiments herein are especially related to an ophthalmic composition incorporating stabilized hypochlorous acid with enhanced shelf life as an ophthalmic preservative. The embodiments herein are especially related to an ophthalmic composition incorporating stabilized hypochlorous acid with enhanced shelf life for use with contact lens solution.

### Description of the Related Art

Ocular infections are incredibly common and can range from being self-limited to severe which can sometimes result in visual loss or blindness. There are numerous causes of eye infections most commonly viral, bacterial and rarely but more devastating fungal. Most ophthalmic antibiotics currently being used are limited in their capacity to only treat certain types of bacterial ocular infections. Also the use of antibiotics regularly leads to antibiotic resistant infections which can result in a super-infection. The antibiotic resistance and subsequent infections routinely can lead to irreversible damage to the ocular structures which may result in corneal scarring and/or glaucoma causing permanent visual loss. Currently there are numerous eye infections resulting from a viral infection which are unable to be treated and only supportive care can be offered. Additionally, the inflammatory process which occurs with ocular infections routinely can be as devastating as the actual infection.

The ophthalmic solutions are administered topically to the eyes. The eyes are extremely sensitive and complex organs. Hence for the ocular safety, it is paramount that the eyedrops and/or contact lens solutions being used are effective yet safe to be used in the eye, such that they do not cause ocular irritation or unwanted, potentially ocular-damaging side effects. As a preservative component of eye drop composition, there are a plurality of preservative compounds currently available. For example bezalkonium chloride, benzethonium chloride, sorbic acid. For contact lens preservative solution preservative example are polyhexamethylene biguanide (PHMB), POLYQUAD, hydrogen peroxide, purite (stabilized chloride dioxide) and the like.

It is observed that bezalkonium chloride or enzethonium chloride have excellent preservative effect. But the side effects are observed, hence limiting the concentration of bezalkonium chloride or enzethonium chloride. Furthermore the additives in the composition change with acidic preservatives because of the chemical reaction. Sorbic acid though less absorptive in eyedrops has significantly less preservative effect. Sorbic acid decreases the pH of the ophthalmic solution. Whereas, hydrogen peroxide and other peroxides as preservatives exhibit excellent washing and sterilization ability of the contact lens storage solution, but because of irritant nature, the peroxides must be completely neutralized.

A plurality of preservatives used in the ophthalmic compositions have toxic side effects, especially when used in higher concentrations. Hypochlorous acid can be utilized as a preservative for ophthalmic compositions. But to be effective as an ophthalmic preservative, stabilized hypochlorous acid should be used in the ophthalmic compositions to maintain longer shelf-life.

Hypochlorous acid is one of the most potent allnatural antimicrobial agents known yet completely non-toxic to humans (mammals). Hypochlorous acid has clinically been shown to kill virtually all known pathogens even prions which cause “Mad Cow Disease”. The hypochlorous acid is naturally produced in the body by white blood cells. Hypochlorous acid also has anti-inflammatory properties which can help with ocular infections and diminish scarring of the cornea with subsequent visual loss. Currently the mainstay of treatment for ocular inflammation is steroids which can have serious side effects such as worsening of the infection (especially fungal or viral infection), thinning of the cornea, as well as causing cataracts and glaucoma. Hypochlorous acid illustrates potent anti-inflammatory benefits without any side effects.

The hypochlorous acid is also produced in a lab using an electrochemical reaction. Unfortunately the hypochlorous acid is very unstable with a short half-life, losing approximately 1-2% potency per day and therefore commercialization of the compositions comprising hypochlorous acid has been challenging.

A plurality of methods are used for stabilizing hypochlorous acid. The methods are (1) adding “stabilizing” agents such as bleach, (2) manipulating the hypochlorous acid molecule by addition of nanoparticles or other micro-particle technology and (3) altering the packaging of the composition. Further by stabilizing hypochlorous acid, a long shelf-life of the composition is achieved enabling the commercialization.

Hypochlorous acid has a plurality of applications in plurality of area. Following are some of the most common applications of hypochlorous acid (1) food processing and handling, (2) sanitization, (3) disinfection, and (4) health care industry. Specifically in the medical field, the hypochlorous acid has been used topically on the skin as a wound cleanser and/or to treat skin infections illustrating its antimicrobial property.

There is a tremendous need for a non-toxic broad-spectrum ophthalmic antimicrobial agent with anti-inflammatory properties. The use of stabilized “Hypochlorous Acid” as an eye drop has been long overdue and has the potential to be the new therapy-of-choice for ocular infections.

Hence there is a need to utilize stabilized hypochlorous acid as an anti-microbial and anti-inflammatory topical ophthalmic eyedrop. There is also a pressing need for an ophthalmic composition comprising stabilized hypochlorous acid as preservative as it has incredibly effective anti-microbial properties without any toxicity.

The above-mentioned shortcomings, disadvantages and problems are addressed herein and which will be understood by reading and studying the following specification.

### OBJECTIVES OF THE EMBODIMENTS

The primary objective of the embodiment herein is to provide an ophthalmic composition comprising stabilized hypochlorous acid as an anti-microbial agent.

Another object of the embodiment herein is to provide an ophthalmic composition comprising stabilized hypochlorous acid as a preservative.

Another object of the embodiment herein is to provide an ophthalmic composition comprising stabilized hypochlorous acid for use with contact lens solutions.

Yet another object of the embodiment herein is to provide an ophthalmic composition comprising stabilized hypochlorous acid which is non-toxic to the human eyes.

Yet another object of the embodiment herein is to provide an ophthalmic composition comprising stabilized hypochlorous acid which has no bacterial resistance.

Yet another object of the embodiment herein is to provide an ophthalmic composition comprising stabilized hypochlorous acid which provides anti-inflammatory benefits.

Yet another object of the embodiment herein is to provide an ophthalmic composition comprising stabilized hypochlorous acid which illustrates antibiotic effect and acts against pathpgens like bacteria, fungi, virus and prions.

Yet another object of the embodiment herein is to provide an ophthalmic composition comprising stabilized hypochlorous acid where the hypochlorous acid acts both as a preservative and as an anti-microbial agent.

These objects and the other advantages of the embodiments herein will become readily apparent from the following detailed description taken in conjunction with the accompanying drawings.

### SUMMARY

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

The following details present a simplified summary of the embodiments herein to provide a basic understanding of the several aspects of the embodiments herein. This summary is not an extensive overview of the embodiments herein. It is not intended to identify key/critical elements of the embodiments herein or to delineate the scope of the embodiments herein. Its sole purpose is to present the concepts of the embodiments herein in a simplified form as a prelude to the more detailed description that is presented later.

The embodiments herein provide an ophthalmic composition comprising stabilized hypochlorous acid as an anti-microbial agent. The embodiments herein further provide an ophthalmic composition comprising stabilized hypochlorous acid as a preservative. The embodiments herein also provide an ophthalmic composition comprising stabilized hypochlorous acid which is non-toxic to human eyes.

According to one embodiment herein, a hypochlorous acid based ophthalmic composition comprises stabilized hypochlorous acid. The stabilized hypochlorous acid is stabilized by a predetermined protocol. The stabilized hypochlorous acid is added in the ophthalmic composition as an anti-microbial agent in a predetermined amount. The stabilized hypochlorous acid is added in the ophthalmic composition as a preservative in a predetermined amount. The stabilized hypochlorous acid is added in the ophthalmic composition in a non-toxic predetermined amount.

According to one embodiment herein, the non toxic predetermined amount of stabilized hypochlorous acid is less than 0.05% v/v.

According to one embodiment herein, the stabilized hypochlorous acid is added in the ophthalmic composition as a preservative in a predetermined amount ranging 0.0005% v/v-0.05% v/v.

According to one embodiment herein, the stabilized hypochlorous acid is added in the ophthalmic composition as an anti-microbial agent after determining free available chlorine (FAC). The free available chlorine (FAC) in the stabilized hypochlorous acid is in a range of 50 ppm-500 ppm.

According to one embodiment herein, the predetermined ophthalmic compositions are selected from a group consisting of eyedrop solution, eyedrop suspension, eyedrop gel and ointment.

According to one embodiment herein, the predetermined ophthalmic compositions consisting of solution, suspension, gel, or ointment are selected to be utilized as an antimicrobial or preservative

These and other aspects of the embodiments herein will be better appreciated and understood when considered in conjunction with the following description and the accompanying drawings. It should be understood, however, that the following descriptions, while indicating preferred embodiments and numerous specific details thereof, are given by way of illustration and not of limitation. Many changes and modifications may be made within the scope of the embodiments herein without departing from the spirit thereof, and the embodiments herein include all such modifications.

### DETAILED DESCRIPTION OF THE EMBODIMENTS

In the following detailed description, a reference is made to the accompanying drawings that form a part hereof, and in which the specific embodiments that may be practiced is shown by way of illustration. The embodiments are described in sufficient detail to enable those skilled in the art to practice the embodiments and it is to be understood that the logical, mechanical and other changes may be made without departing from the scope of the embodiments. The following detailed description is therefore not to be taken in a limiting sense.

The embodiments herein provide an ophthalmic composition comprising stabilized hypochlorous acid as preservative. The embodiments herein further provide an ophthalmic composition comprising stabilized hypochlorous acid as anti-microbial agent. The embodiments herein also provide an ophthalmic composition comprising stabilized hypochlorous acid which is not toxic to the human eyes.

According to one embodiment herein, a hypochlorous acid based ophthalmic composition comprises stabilized hypochlorous acid. The stabilized hypochlorous acid is stabilized by a predetermined protocol. The stabilized hypochlorous acid is added in the ophthalmic composition as a preservative in a predetermined amount. The stabilized hypochlorous acid is added in the ophthalmic composition as an anti-microbial agent in a predetermined amount. The stabilized hypochlorous acid is added in the ophthalmic composition in a non-toxic predetermined amount.

According to one embodiment herein, the non toxic predetermined amount of stabilized hypochlorous acid is in a range of 0.0002% v/v-0.03% v/v.

According to one embodiment herein, the stabilized hypochlorous acid is added in the ophthalmic composition as a preservative in a predetermined amount ranging 0.0005% v/v-0.02% v/v.

According to one embodiment herein, the stabilized hypochlorous acid is added in the ophthalmic composition as an anti-microbial agent after determining free available chlorine (FAC). The free available chlorine (FAC) in the stabilized hypochlorous acid is in a range of 100 ppm-500 ppm.

According to one embodiment herein, the predetermined ophthalmic compositions are selected from a group consisting of anti-microbial eye drops, eye cleansing drops, ophthalmic lubricants and contact lens solutions.

According to one embodiment herein, the stabilized hypochlorous acid is used with a concentration being in the range of 0.002% v/v-0.03% v/v. The ophthalmic composition comprising stabilized hypochlorous acid, where the hypochlorous acid acts both as preservative and anti-microbial agent.

According to one embodiment herein, use of stabilized hypochlorous acid in a range of 0.0005% v/v-0.02% v/v as a preservative for eye drop composition. The use of stabilized hypochlorous acid illustrates no toxicity. Hence the use of stabilized hypochlorous acid as preservative and as an ophthalmic anti-microbial composition illustrates no side effects.

According to one embodiment herein, the composition for the eye drops comprises hypochlorous acid comprising free available chlorine (FAC) in a range of 100 ppm to 500 ppm. Depending on the therapeutic requirement, a specific free available chlorine is determined.

According to one embodiment herein, the ophthalmic composition comprising hypochlorous acid as preservative comprises hypochlorous acid in a range of 0.0005% v/v to 0.02% v/v. The specific range is predetermined based on the eye drop formulation and requirement.

According to one embodiment herein, the hypochlorous acid can also be utilized as contact lens solution in predetermined concentration.

According to one embodiment herein, the hypochlorous acid is manufactured and stabilized by a predetermined protocol. The hypochlorous acid is stored in a suitable container for topical ophthalmic application. Before packing the hypochlorous acid in the container, the container is flushed with an inert gas to remove any other air/gas and replace the gap with inert gas prior sealing.

It is to be understood that the phraseology or terminology employed herein is for description and not of limitation. Therefore, while the embodiments herein have been described in terms of preferred embodiments, those skilled in the art will recognize that the embodiments herein can be practiced with modification within the spirit and scope of the appended claims.

Although the embodiments herein are described with various specific embodiments, it will be obvious for a person skilled in the art to practice the invention with modifications. However, all such modifications are deemed to be within the scope of the claims.

It is also to be understood that the following claims are intended to cover all of the generic and specific features of the embodiments described herein and all the statements of the scope of the embodiments which as a matter of language might be said to fall there between.

What is claimed is:

1. A hypochlorous acid based ophthalmic composition, the composition comprises:
stabilized hypochlorous acid, and wherein the stabilized hypochlorous acid is stabilized by a predetermined protocol, and wherein the stabilized hypochlorous acid is added in the ophthalmic composition as an anti-microbial agent in a predetermined amount, and wherein the stabilized hypochlorous acid is added in the ophthalmic composition as a preservative in a predetermined amount, and wherein the stabilized hypochlorous acid is added in the ophthalmic composition to be utilized for contact lens solution and wherein the stabilized hypochlorous acid is added in the ophthalmic composition as a non-toxic component in a predetermined amount.

2. The composition according to claim 1, wherein the predetermined amount of stabilized hypochlorous acid added as the non-toxic component less than 0.05% v/v.

3. The composition according to claim 1, wherein the stabilized hypochlorous acid is added in the ophthalmic composition as a preservative in a predetermined amount ranging 0.0005% v/v-0.05% v/v.

4. The composition according to claim 1, wherein the stabilized hypochlorous acid is added in the ophthalmic composition as an anti-microbial agent after determining free available chlorine (FAC), and wherein the free available chlorine (FAC) in the stabilized hypochlorous acid is in a range of 50 ppm-500 ppm.

5. The composition according to claim 1, wherein the predetermined ophthalmic compositions are selected from a group consisting of anti-microbial eye drops, eye cleansing drops, ophthalmic lubricants and contact lens solutions.

6. The composition according to claim 1, wherein the predetermined ophthalmic compositions are selected from a group consisting of eyedrop solution, eyedrop suspension, eyedrop gel and ointment.

7. The composition according to claim 1, wherein the predetermined ophthalmic compositions consisting of solution, suspension, gel, or ointment are selected to be utilized as an antimicrobial or preservative.
